(19) United States
(12) Patent Application Publication
(10) Pub. No.: US 2025/0009533 A1
(43) Pub. Date: Jan. 9, 2025

(54) DESIGN AND FABRICATION OF PATIENT-SPECIFIC STIMULI-RESPONSIVE CARDIOVASCULAR STENTS FOR CORONARY ARTERY SPASM TREATMENT TO ELIMINATE POST-SURGICAL INTERVENTIONS

(71) Applicant: Qatar Foundation for Education, Science and Community Development, Doha (QA)

(72) Inventors: Muammer Koc, Doha (QA); Ans Al Rashid, Doha (QA)

(21) Appl. No.: 18/762,014

(22) Filed: Jul. 2, 2024

Related U.S. Application Data
(60) Provisional application No. 63/525,289, filed on Jul. 6, 2023.

(51) Int. Cl. A61F 2/82; A61L 31/02; A61L 31/06; B33Y 70/00; B33Y 80/00

(52) U.S. Cl. CPC A61F 2/82; A61L 31/022; A61L 31/06; B33Y 70/00; B33Y 80/00; A61F 2210/0014; A61F 2210/009; A61F 2240/001

(57) ABSTRACT

Example systems, methods, and apparatus are disclosed herein for a design and fabrication of patient-specific stimuli-responsive cardiovascular stents for coronary artery spasm treatment to eliminate post-surgical interventions. The stimuli-responsive material may be a magnetically-induced shape memory nanoparticle such as Hematite dispersed in PLA. Such Hematite may be at a concentration of 10 wt % to 20 wt %.

### PRIORITY CLAIM AND CROSS-REFERENCE TO RELATED APPLICATIONS

The present application claims the benefit of and priority to U.S. Provisional Application No. 63/525,289 filed Jul. 6, 2023, which is incorporated herein by reference in its entirety.

### BACKGROUND

Stents for coronary artery treatment have been an outstanding medical science achievement. Coronary artery spasm arises through the thickening of the blood vessels, which reduces or completely blocks the blood flow due to cholesterol or fatty deposits. Initially deployed stents were composed of metals and alloys due to their strength and ability to reduce the strut thickness without compromising its structural integrity. However, metallic stents gave rise to the problem of neointimal hyperplasia. Another issue related to these stents is caused by the development of fatty plaque in the stent region, reducing the lumen space, termed in-stent restenosis. Initially, oral drugs were used to treat restenosis due to metallic stents. This treatment was not sufficient, and surface coated stents with drugs were employed to serve the cause. Drug-eluting stents slowly release a drug to avoid neointimal hyperplasia. However, drug coating depletes over time, directly interacting blood cells with stent struts and reducing endothelial regenerations, leading to late stent thrombosis (ST). Second-generation DES provided better physio-mechanical properties and biocompatibility, but very late stent thrombosis remained.

Coronary artery disease is liable for 31% of all passings around the world, putting the health system and economy of the countries at risk. Regardless of the broad advancements in cardiovascular imaging and helpful regimens, the extensiveness of the disease still rises, which consequently increases the number of patients suffering from myocardial infarction. To prevent and control such strokes, stents are widely used to open the arteries and supply enough blood to the heart. Similarly, given the importance of the trachea in the human body, its vulnerability must be protected. Any obstruction of the airway has the potential of fatally endangering the life of a person. Nonetheless, due to its fragile nature, if the trachea is once injured, it can become difficult to fix it. One way of doing so is endotracheal stent intervention, in which the diameter of the trachea is reshaped by implanting a stent.

The prime objective of the coronary stent is to keep up with the vessel's patency. Stents are designed to satisfy the vessel's compatibility, adaptability, and diameter with low chances of thrombosis that occurs due to endothelial dysfunction. Initially deployed stents were composed of metals or alloys (stainless steel, cobalt-chromium alloys) due to their strength and ability to reduce the strut thickness without compromising the stent's structural integrity. However, alloy stents gave rise to the problem of neointimal hyperplasia, the migration of muscular cells from tunica media to tunica intima resulting in reduced lumen space. As discussed above, another issue related to these stents is caused by the development of fatty plaque in the stent region, again decreasing the lumen space and termed in-stent restenosis. Initially, oral drugs were used to treat restenosis due to metallic stents. This treatment was insufficient, and surface-coated stents with drugs were employed to serve the cause. P. Serruys developed first-generation drug-eluting stents (DES), slowly releasing a drug to avoid neointimal hyperplasia. However, drug coating depletes over time, resulting in direct interaction of blood cells with stent struts and reduced endothelial regenerations, leading to late stent thrombosis (ST). Second-generation DES provided better physio-mechanical properties and biocompatibility, but very late stent thrombosis remained. Vishnu et al., “Perspectives on Smart Stents with Sensors: From Conventional Permanent to Novel Bioresorbable Smart Stent Technologies” (2020), which is hereby incorporated by reference in its entirety, presents an insight into the above-mentioned cardiovascular stent complications. Generally, secondary interventions are made to treat these problems, which causes additional costs to healthcare and increased risk to the patient. Besides the design, fabrication, and implantation of stents, post-stenting complications can lead to mortalities and must be addressed in a timely manner.

Polymer or metallic stents with bioresorbable properties, termed bioresorbable stents (BRS), have arisen recently to avoid the problems associated with permanent stents. Several studies reported the excellent biocompatibility of stents in in-vitro and in-vivo conditions; however, the successful and in-vivo implementation of stents requires multi-faceted considerations. To date, metallic alloy-based stents are employed to treat coronary artery spasms. Few studies reported testing polymers and their composites; however, these materials are not fully developed and explored for commercial utilization. Besides, advanced manufacturing processes, such as 3D Printing (3DP), can provide freedom to produce patient-specific products with in-house fabrication. Utilization of these processes in medical emergencies and uncertain situations (like COVID), when delayed supply chains can cause mortalities, can provide rapid, patient-specific, and reliable solutions.

Although a metallic stent may provide the desired mechanical properties, clinical issues related to these materials led to the exploration of polymers as potential materials. Primary shortcomings associated with polymers are lack of radiopacity, lower radial stresses, and plasticity. As polymers exhibit inferior mechanical properties, thicker struts can avoid elastic recoil and withstand radial stresses. Poly L-lactic acid (PLLA), a FDA approved biomaterial, has been utilized recently to serve the cause. Several attempts have been made to fabricate stents using different polymeric and composite materials, including; polylactic acid (PLA) with chitosan and paclitaxel coating, polyurethane (PU) added to polyester, PLLA, polycaprolactone (PCL) with wireless pressure sensors. PLA is a thermoplastic polyester obtained from biodegradable resources and exhibits excellent mechanical and thermal properties, good processability, and low environmental impact. PLA is globally accepted as biodegradable because it originates from plants and has proven safe for biomedical applications and food industries. Therefore, these are being explored in tissue engineering, biomedical implants, and food packaging sectors for their performance.

In addition, stents made of high-paramagnetic iron oxide nanoparticles encourage functionalized intervention devices for different biomedical applications, including myocardial infarction conditions and other coronary artery diseases. High-paramagnetic nanoparticles that are common in the research community are magnetite (Fe3O4) and hematite (α-Fe2O3), which are usually coated with polymers. The coating is used for good dispersion of the particles in the mixture for stable samples, allowing the conjugation of magnetic-sensitive ligands and increasing the magnetization properties. Moreover, as magnetite and hematite display high paramagnetic properties, the remote actuation of their mass structure is conceivable through an external magnetic field for cardiovascular therapy.

Magnetic-sensitive nanoparticles have been widely adopted for various applications, especially biomedical (drug delivery, magnetic resonance imaging, etc.), storage media, and photochemical applications. Specifically, hematite (α-Fe2O3) is one of the most common magnetic sensitive oxides which possess unique electrical and magnetic properties relying upon the Fe+2 ion. Due to their attractive structural, chemical, thermal and suitable surface characteristics, superparamagnetic nanoparticles are being explored for biomedical applications. Several methods are available to synthesize various nanocomposites with desired properties. However, the technique used for biomedical applications, especially functional cardiovascular stents, is still under development. The interest in metal nanoparticle research is increasing nowadays, especially in plant tissues, plant byproducts, and other parts of biological plants. The magnetic behavior of PLA/Fe2O3 is studied by Zhang et al., “Design of 4D Printed Shape-Changing Tracheal Stent and Remote Controlling Actuation” (2021), which is hereby incorporated by reference in its entirety, where the shape-changing behavior of the composites with time was investigated, which ultimately satisfies the shape-changing properties of polymer composites with time.

Finally, to achieve the facile fabrication of complex stent structures with minimal waste and facilitate rapid fabrication, 3D printing (3DP) processes can be explored and adopted. These processes have proved their potential in recent years over many applications. Therefore, as a preliminary validation of proof of concept to develop such materials for 3DP processes, stimuli-responsive stents are required that can be actuated remotely through a magnetic field and adjusted according to the requirements. In this study, stimuli-responsive polymer nanocomposites were synthesized by doping α-Fe2O3 into polylactic acid (PLA) through a solvent-casting approach. The nanocomposites were then characterized using Fourier transform infrared spectroscopy (FTIR), thermogravimetric analysis (TGA), and differential scanning calorimetry (DSC). Finally, electromagnetic capability using magnetic hysteresis analysis is also performed to assess actuation under magnetic stimulus in a controlled environment.

Therefore, there is a need for designing innovative cardiovascular stents using 3D printed polymer nanocomposites with desired mechanical and biological properties. There is further a need for a process that includes adequate stent design, polymer and reinforcement selection, material synthesis, characterization of stimuli-responsive biocompatible polymer nanocomposites, 3D printing (3DP) of stents, testing, and validation. These stents will need to possess significant mechanical strength to perform the functions properly with structural integrity and biocompatibility. There is also a need to design a compliant structure for the stent with shape memory characteristics to retain the desired shape under varying conditions. Additionally, these stents will need to be incorporated with biocompatible piezoelectric materials (Polyvinylidene fluoride/iron oxide) to generate magnetic stimuli from external sources to produce the desired shape and size changes. These structures need flexible and rapid fabrication for different patients with higher dimensional accuracy; therefore, a facile manufacturing process (3DP) can be used to fabricate them rapidly and precisely. The disclosed invention will possess a compliant design for the end-user by eliminating the post-angioplasty in case of any deformation or atrial contraction/expansion and allowing the external control of the stent structure for size adjustments.

### SUMMARY

Example systems, methods, and apparatus are disclosed herein for the design and fabrication of patient-specific stimuli-responsive cardiovascular stents for coronary artery spasm treatment to eliminate post-surgical interventions.

In light of the disclosure herein, and without limiting the scope of the invention in any way, in a first aspect of the present disclosure, which may be combined with any other aspect listed herein unless specified otherwise, there is provided a patient-specific stimuli-responsive cardiovascular stent for coronary artery spasm treatment to eliminate post-surgical interventions.

In a second aspect of the present disclosure, which may be combined with any other aspect listed herein unless specified otherwise, the cardiovascular stent comprises a magnetic nanoparticle configured to enable magnetically induced shape memory effects.

In a third aspect of the present disclosure, which may be combined with any other aspect listed herein unless specified otherwise, the magnetic nanoparticle comprises Hematite (α-Fe2O3).

In a fourth aspect of the present disclosure, which may be combined with any other aspect listed herein unless specified otherwise, the concentration of Hematite is at least 10 wt %.

In a fifth aspect of the present disclosure, which may be combined with any other aspect listed herein unless specified otherwise, the concentration of Hematite is at least 15 wt %.

In a sixth aspect of the present disclosure, which may be combined with any other aspect listed herein unless specified otherwise, the concentration of Hematite is at least 20 wt %.

In a seventh aspect of the present disclosure, which may be combined with any other aspect listed herein unless specified otherwise, the cardiovascular stent is configured to have a desired magnetically induced shape memory position for a coronary artery spasm treatment.

In an eighth aspect of the present disclosure, which may be combined with any other aspect listed herein unless specified otherwise, there is provided a method of fabricating a patient-specific stimuli-responsive cardiovascular stent for coronary artery spasm treatment to eliminate post-surgical interventions.

In a ninth aspect of the present disclosure, which may be combined with any other aspect listed herein unless specified otherwise, the method comprises dissolving PLA in dichloromethane (DCM) using a mechanical mixer; adding a concentration of magnetic sensitive α-Fe2O3 nanoparticles to the PLA/DCM solution; mechanically stirring the solution for 4 hours at a speed of between 1300-1500 rpm; heating the solution to 40° C. with continuous stirring to remove the solvent (DCM) from the mixture; laying the PLA/α-Fe2O3 nanocomposites on a flat surface to remove the entrapped solvent; and obtaining nanocomposites in film form.

In another aspect of the present disclosure, which may be combined with any other aspect listed herein unless specified otherwise, the method further comprises forming a stent by additive manufacturing using the nanocomposites.

In another aspect of the present disclosure, which may be combined with any other aspect listed herein unless specified otherwise, the concentration of magnetic sensitive Hematite (α-Fe2O3) is 10 wt %.

In another aspect of the present disclosure, which may be combined with any other aspect listed herein unless specified otherwise, the concentration of magnetic sensitive Hematite (α-Fe2O3) is 15 wt %.

In another aspect of the present disclosure, which may be combined with any other aspect listed herein unless specified otherwise, the concentration of magnetic sensitive Hematite (α-Fe2O3) is 20 wt %.

In another aspect of the present disclosure, which may be combined with any other aspect listed herein unless specified otherwise, the method further comprises configuring the stent to have a desired magnetically induced shape memory position.

In another aspect of the present disclosure, which may be combined with any other aspect listed herein unless specified otherwise, there is provided a method of using a patient-specific stimuli-responsive cardiovascular stent for coronary artery spasm treatment to eliminate post-surgical interventions.

In another aspect of the present disclosure, which may be combined with any other aspect listed herein unless specified otherwise, the method comprises determining a desired configuration of the stimuli-responsive cardiovascular stent based on a physiology of a patient; and producing a stimuli-responsive cardiovascular stent based on the desired configuration. In another aspect of the present disclosure, which may be combined with any other aspect listed herein unless specified otherwise, the cardiovascular stent is produced by a process of additive manufacturing.

In another aspect of the present disclosure, which may be combined with any other aspect listed herein unless specified otherwise, the process of additive manufacturing occurs within the vicinity of the patient.

In another aspect of the present disclosure, which may be combined with any other aspect listed herein unless specified otherwise, the cardiovascular stent comprises a PLA/α-Fe2O3 nanocomposite.

In another aspect of the present disclosure, which may be combined with any other aspect listed herein unless specified otherwise, the concentration of α-Fe2O3 in the PLA/α-Fe2O3 nanocomposite is at least 10 wt %.

In another aspect of the present disclosure, which may be combined with any other aspect listed herein unless specified otherwise, the concentration of α-Fe203 in the PLA/α-Fe2O3 nanocomposite is at least 15 wt %.

In another aspect of the present disclosure, which may be combined with any other aspect listed herein unless specified otherwise, the concentration of α-Fe2O3 in the PLA/α-Fe2O3 nanocomposite is at least 20 wt %.

In another aspect of the present disclosure, which may be combined with any other aspect listed herein unless specified otherwise, the method further comprises providing the cardiovascular stent to the patient; and actuating the cardiovascular stent by applying a magnetic field near the cardiovascular stent.

In another aspect of the present disclosure, any of the structure, functionality, and alternatives disclosed in connection with any one or more of FIGS. 1 to 4 may be combined with any other structure, functionality, and alternatives disclosed in connection with any other one or more of FIGS. 1 to 4.

In light of the present disclosure and the above aspects, it is therefore an advantage of the present disclosure to provide users with a design and fabrication of patient-specific stimuli-responsive cardiovascular stents for coronary artery spasm treatment to eliminate post-surgical interventions.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a graph of FTIR spectrum of PLA/α-Fe2O3 Nanocomposites, according to an example embodiment of the present disclosure.

FIG. 2 is a graph of DSC analysis of PLA/α-Fe2O3 Nanocomposites, according to an example embodiment of the present disclosure.

FIG. 3 is a graph of TGA analysis of PLA/α-Fe2O3 Nanocomposites, according to an example embodiment of the present disclosure.

FIG. 4 is a graph of Magnetic Sensitivity of PLA/α-Fe2O3 Nanocomposites, according to an example embodiment of the present disclosure.

### DETAILED DESCRIPTION

Methods, systems, and apparatus are disclosed herein for the design and fabrication of patient-specific stimuli-responsive cardiovascular stents for coronary artery spasm treatment to eliminate post-surgical interventions.

While the example methods, apparatus, and systems are disclosed herein the design and fabrication of patient-specific stimuli-responsive cardiovascular stents for coronary artery spasm treatment to eliminate post-surgical interventions, it should be appreciated that the methods, apparatus, and systems may be operable for other medical conditions.

The disclosed invention presents preliminary findings on the performance of the stimuli-responsive PLA/α-Fe2O3 nanocomposites under magnetic stimulus with varying concentrations of hematite (α-Fe2O3) nanoparticles. The effect of varying nanoparticle concentrations on the structural and thermal properties was examined using Fourier transform infrared spectroscopy (FTIR), thermogravimetric analysis (TGA), and differential scanning calorimetry (DSC). Subsequently, the magnetic field with various intensities was generated using a vibrating sample magnetometer in a controlled environment. The resulting deformation rate of the nanocomposites was observed in response to changing magnetic fields. The results revealed that the PLA/α-Fe2O3 nanocomposites could be potential materials for biomedical applications, i.e., cardiovascular stents, which can provide the desired stimulus to treat post-stenting complications without the need for secondary surgical procedures. In future studies, the synthesized PLA/α-Fe2O3 nanocomposites will be adopted for 3D printing (3DP) processes to develop patient-specific cardiovascular stents and to evaluate their biomechanical performance.

The cardiovascular stent may be produced by a process of 3DP on-site at a health care facility based on a measured physiology of a cardiovascular patient. For example, the length and diameter of a specific stricture caused by an arterial blockage may be measured and used to determine the dimensions of the cardiovascular stent produced by 3DP. Such a system and method may be advantageous when supply chain restrictions cause a delay or lack of available cardiovascular stents. Another advantage to the systems and methods is increased efficaciousness because of the customized patient-specific dimensions of the cardiovascular stents. Another advantage is the resilience of the cardiovascular stents based on their shape-memory characteristics and magnetic actuation. Certain concentrations of shape-memory nanoparticle may be advantageous based on the specific needs of a patient, location of the artery and blockage, the strength of the magnetic field used to actuate the cardiovascular stent, and any other possible variables.

### Preparation and Materials

Synthesis of PLA α-Fe2O3 Nanocomposite: The solvent casting approach was utilized to synthesize the polymer nanocomposites. First, PLA pallets were dissolved in dichloromethane (DCM) using a mechanical mixer. Varying concentrations (10 wt %, 15 wt %, and 20 wt %) of magnetic sensitive α-Fe2O3 nanoparticles were added to the PLA/DCM solution and mechanically stirred for 4 hours at a speed of 1300-1500 rpm. Subsequently, the solution was heated to 40° C. with continuous stirring to remove the solvent (DCM) from the mixture. Finally, the PLA/α-Fe2O3 nanocomposites were laid on a flat surface to remove the entrapped solvent, and nanocomposites were obtained in film form.

PLA α-Fe2O3 Nanocomposite Characterization: FTIR analysis was performed in transmittance mode to gain chemical insights into the stimuli-responsive nanocomposites using (Thermo Scientific Nicolet iS50 FT-IR) spectrometer equipped with an attenuated total reflectance (ATR) sampling accessory with a diamond crystal plate. The thermal properties of the nanocomposite were characterized using thermogravimetric analysis (TGA) and differential scanning calorimetry (DSC). For TGA, the analyzer STA7300 (from Hitachi company) was used, with the temperature range from room temperature to 700° C. at a rate of 10° C./min under a nitrogen gas environment to avoid oxidation. For DSC, the SF1942 instrument (from Mettler Toledo company, Switzerland) was used at a temperature range from room temperature to 200° C. at a rate of 10° C./min.

### Magnetic Stimuli Response

Using a magnetic field, the PLA/α-Fe2O3 nanocomposites were also characterized for their response under remote actuation. A vibrating sample magnetometer (VSM, Lakeshore 340, USA) provided the required magnetic intensity at room temperature (300 K) by saturating the sample in a magnetic field of strength 8T. The PLA/α-Fe2O3 nanocomposites were converted to powder form with an average particle size of 50-60 μm to be fed into the manometer.

### Fourier Transform Infrared Spectroscopy (FTIR)

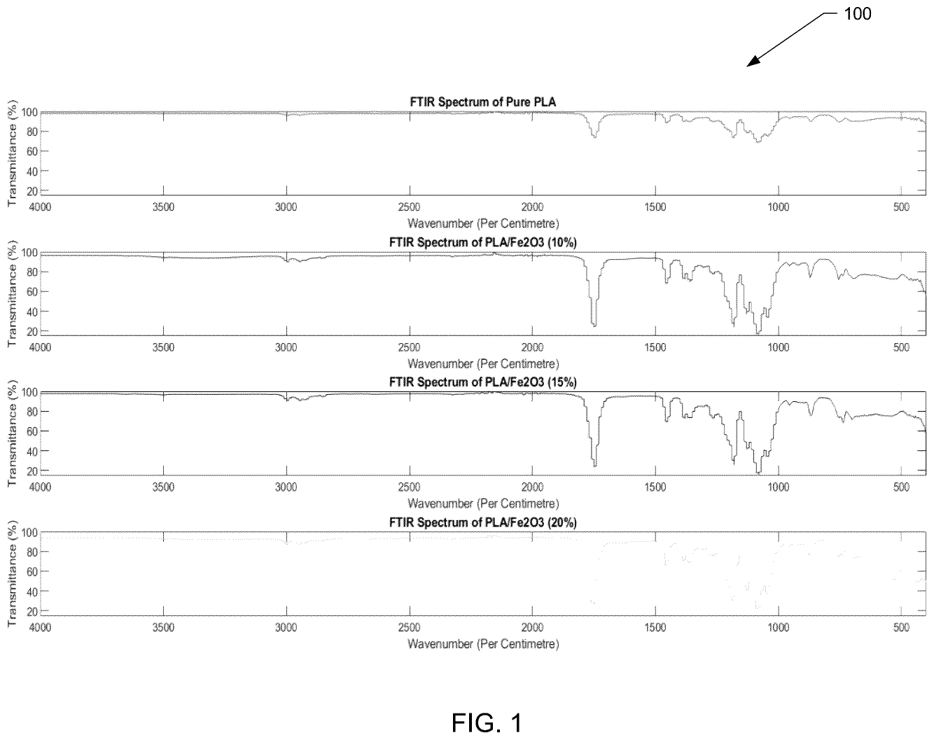

FTIR analysis can provide significant insight into the structure of polymer nanocomposites by identifying the molecular interactions. Considerable changes (band shifting or broadening) can be observed between pure polymer and polymer nanocomposite spectra in the case of chemical interactions (i.e., hydrogen bonding or dipolar interaction). A characteristic peak at a lower wavenumber corresponds to a strong interaction between polymer and nanoparticles. FIG. 1 shows the FTIR spectra of pure PLA and synthesized PLA-reinforced hematite (α-Fe2O3) nanoparticles 100. Three characteristic peaks were observed for pure PLA, two between 1000 cm-1 to 1250 cm-1 and one around 1750 cm-1, corresponding to stretching of —OH, —C═O, and C—O groups. For PLA/α-Fe2O3 nanocomposites, the stretching in peaks between 1000 cm-1 to 1250 cm-1 and 1750 cm-1 is noticeable due to the bending vibration of hydroxyl groups and C═O stretching vibration, respectively. Similar effects were also observed by incorporating carbon black and carbon nanotubes into PLA. Finally, the characteristic peak for α-Fe2O3 nanoparticles at around 3000 cm-1 was not reasonably detected due to the low concentration of α-Fe2O3 nanoparticles within the PLA.

### DSC & TGA Analysis

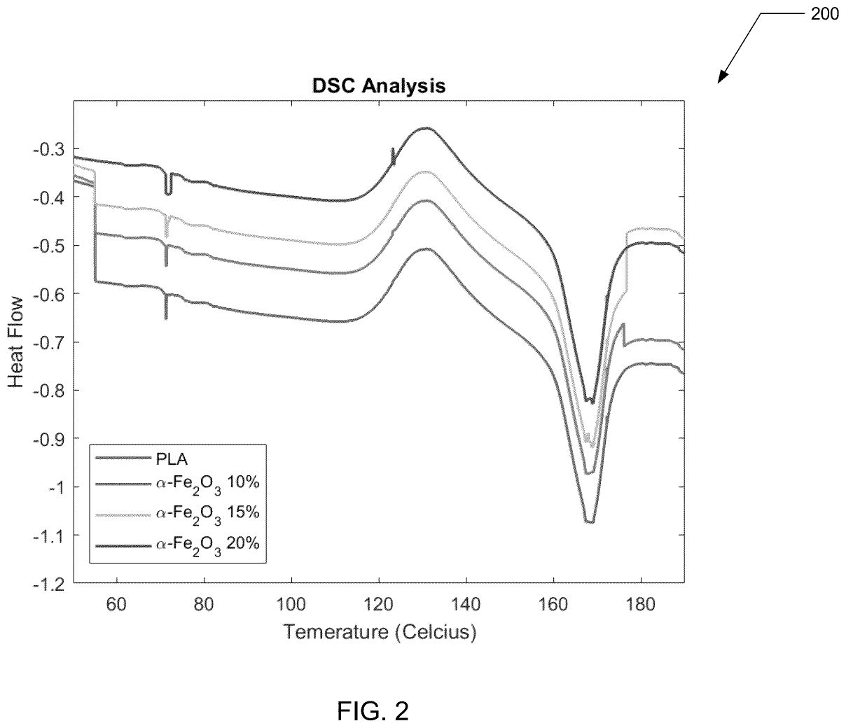

The stimuli-responsive PLA/α-Fe2O3 nanocomposites were characterized using DSC and TGA. FIG. 2 is a graph of DSC analysis of PLA/α-Fe2O3 Nanocomposites DSC results 200. FIG. 2 shows that at around 71° C., there is a sharp valley in the curve, indicating the glass transition temperature of the compound (and the shape that the shape memory would be if it were 3D-printed). The slope of the curve begins to increase at about 110° C., indicating the crystallization of the PLA/α-Fe2O3 polymer nanocomposites. The gradient next takes a steep hit starting at about 153° C., showing that the melting point of the composites has been reached. However, the heat flow is increased due to the reinforcement of α-Fe2O3 to PLA. The first drop in heat flow at 71.38° C. for pure PLA corresponds to the glass transition temperature. In addition, a significant increase in the slope is observed at 131° C., referring to the crystallization temperature. At the melting temperature, there is a substantial drop in the heat flow, and the drop at this point is consistent for all the materials under consideration. DSC curves of all PLA/α-Fe2O3 concentrations have produced the same pattern, showing that the addition of α-Fe2O3 does not have a significant effect on these temperatures, although the heat flow is increased by almost 54% with 20% reinforcement. The glass transition, crystallization, and melting temperatures of each material are reported in Table 1 (below).

TABLE 1
Phase change temperature of PLA/α-Fe2O3 Nanocomposites.

| | Glass Transition | Crystallization | Melting |
|---|---|---|---|
| PLA/Composite | Temperature (° C.) | Temperature (° C.) | Temperature (° C.) |
| Pure PLA | 71.38 | 131.00 | 168.66 |
| PLA/α-Fe₂O₃10% | 72.34 | 132.32 | 169.4 |
| PLA/α-Fe₂O₃15% | 73.2 | 133.4 | 170.3 |
| PLA/α-Fe₂O₃20% | 74 | 134.1 | 171.2 |

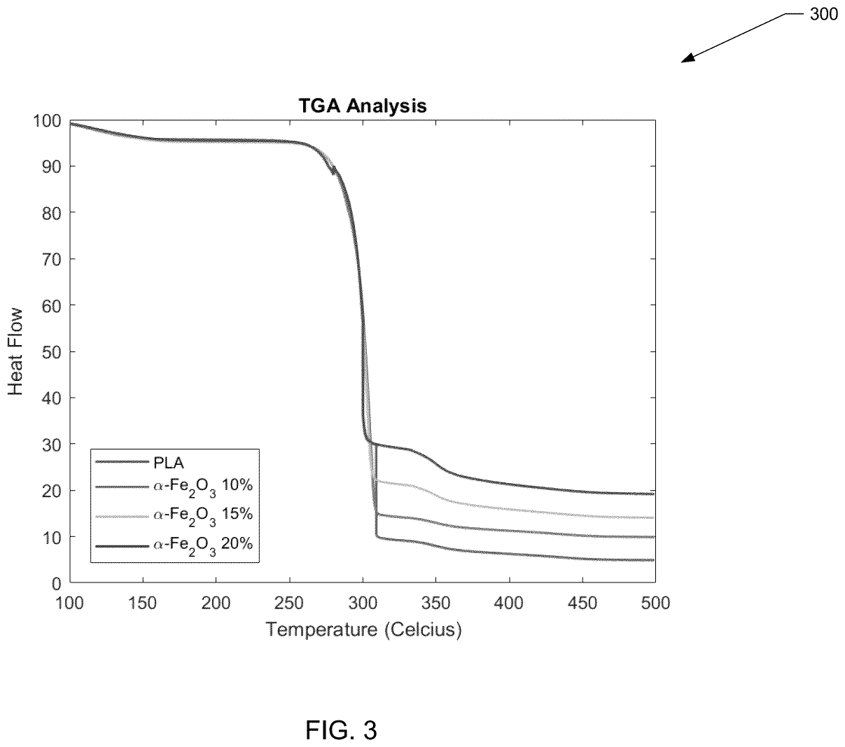

The thermal stability of the samples was analyzed using TGA. TGA was performed between 0° C. to 500° C. at a temperature ramp of 10° C./min. FIG. 3 is a graph of TGA analysis of PLA/α-Fe2O3 Nanocomposites 300. FIG. 3 shows the results for pure PLA and PLA/α-Fe2O3 nanocomposites with increasing weight composition (10%, 15%, and 20%), respectively. As seen in the TGA curves, there is a gradual mass loss followed by a steep loss starting at 280° C. This abrupt mass loss is attributed to the loss of PLA from the polymer nanocomposites due to heating. The samples begin to degrade at around 309° C. The melting temperatures of all the PLA/α-Fe2O3 composites increase with increasing composition and as compared with the neat PLA. This is in line with the observation that the melting point of the mixture would be higher than that of the pure substance. Similar DSC and TGA results were observed in the literature by incorporating metallic nanoparticles into the PLA matrix.

### Magnetic Stimuli Response

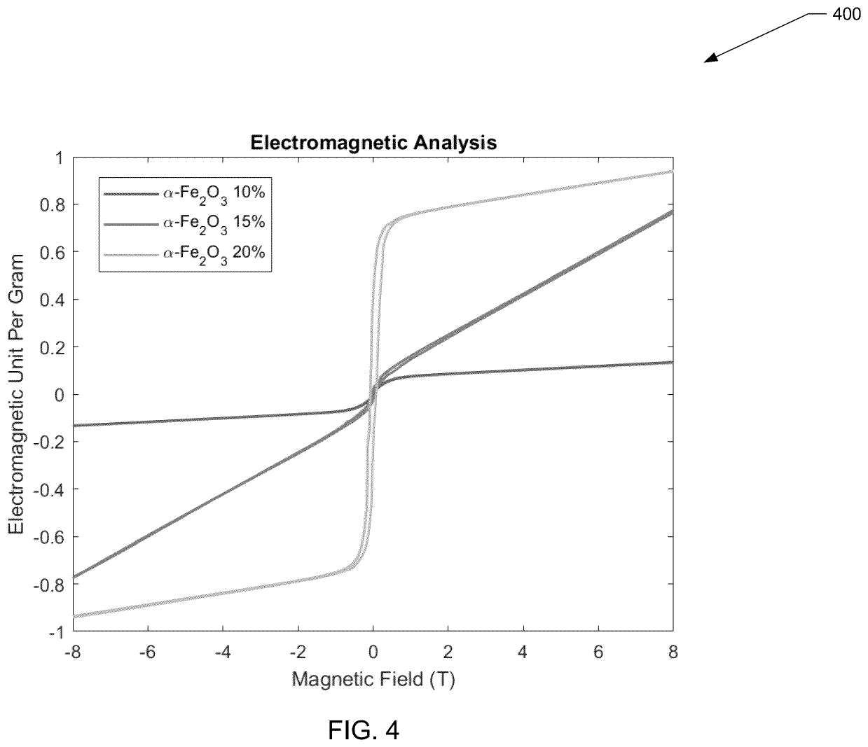

Electromagnetic capability analysis of the synthesized PLA/α-Fe2O3 nanocomposites was also performed to produce magnetic hysteresis curves, as shown in FIG. 4. FIG. 4 is a graph of Magnetic Sensitivity of PLA/α-Fe2O3 Nanocomposites 400. These curves can be quantified using the electromagnetic unit per gram, which indicates the electromagnetic abilities of the tested compound per gram of its weight. The hysteresis curves revealed that the increase in α-Fe2O3 nanoparticles concentration also increases the electromagnetic capabilities of the PLA/α-Fe2O3 nanocomposites. There is visible hysteresis in the compound with the highest α-Fe2O3 concentration and lesser significance at lower concentrations. The space inside the hysteresis loop indicates the magnetic field required to align the ferromagnetic particles with a magnetic field when an external magnetic field is applied. Thus, the maximum force for magnetization is needed for PLA/α-Fe2O3 nanocomposites at 20% of α-Fe2O3 concentration. In addition, there is a more significant electromagnetic capability increase when the concentration of α-Fe2O3 is increased from 15% to 20%, compared to an increase from 10% to 15%. However, the hysteresis curves exhibit properties of soft ferromagnets—i.e., low remanence, lesser coercive force, and low hysteresis losses. Though iron oxide in the mass structure has ferromagnetic characteristics, it also shows superparamagnetic behavior for single-area nanoparticles at the micro and nanoscale. For example, the movement of the sensitive iron oxide particles can easily be adjusted by a remotely applied magnetic field, which generates a net magnetism of the sample. The sample no longer has its polarization if the applied attractive field is turned off.

The disclosed invention uses the Hematite (α-Fe2O3) nanoparticles at different concentrations to synthesize the magnetic responsive PLA/α-Fe2O3 nanocomposites. The PLA/α-Fe2O3 nanocomposites were characterized for their thermochemical and magnetic properties. The results reveal increased heat flow characteristics with the addition of α-Fe2O3 nanoparticles to PLA. The magnetic stimuli response of the PLA/α-Fe2O3 nanocomposites showed that the reinforcement of Hematite (α-Fe2O3) nanoparticles significantly affects the PLA's magnetic properties. The magnetic capability of PLA/α-Fe2O3 nanocomposites demonstrated a drastic increase from 15% to 20%, with an approximate value of 0.82 emu/gram. The reported modification of PLA with α-Fe2O3 nanoparticles suggested the likelihood of directing future work into further analysis of applications where remote actuation is desired. Further studies are required to ensure the effectiveness of its application in the biomedical field by designing and 3D printing of remotely actuated devices to evaluate their deformation and recovery against the magnetic field.

### Conclusion

The term vicinity is used herein to describe a relationship between elements. Vicinity may be interpreted as with the same room, within the same building, within the same campus, or any other reasonable interpretation based on the context. For example, and not limiting the scope of any of the claims, the vicinity of a patient in a hospital may reasonably within the same hospital.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A patient-specific stimuli-responsive cardiovascular stent for coronary artery spasm treatment to eliminate post-surgical interventions comprising a magnetic nanoparticle configured to enable magnetically induced shape memory effects.

2. The stent of claim 1, wherein the magnetic nanoparticle comprises Hematite (α-Fe₂O₃).

3. The stent of claim 2, wherein the concentration of Hematite is at least 10 wt %.

4. The stent of claim 2, wherein the concentration of Hematite is at least 15 wt %.

5. The stent of claim 2, wherein the concentration of Hematite is at least 20 wt %.

6. The stent of claim 1, wherein the stent is configured to have a desired magnetically induced shape memory position for a coronary artery spasm treatment.

7. A method of fabricating a patient-specific stimuli-responsive cardiovascular stent for coronary artery spasm treatment to eliminate post-surgical interventions comprising:
dissolving PLA in dichloromethane (DCM) using a mechanical mixer;
adding a concentration of magnetic sensitive α-Fe₂O₃ nanoparticles to the PLA/DCM solution;
mechanically stirring the solution for 4 hours at a speed of between 1300-1500 rpm;
heating the solution to 40° C. with continuous stirring to remove the solvent (DCM) from the mixture;
laying the PLA/α-Fe₂O₃ nanocomposites on a flat surface to remove the entrapped solvent; and
obtaining nanocomposites in film form.

8. The method of claim 7, further comprising:
forming a stent by additive manufacturing using the nanocomposites.

9. The method of claim 8, wherein the concentration of magnetic sensitive Hematite (α-Fe2O3) is 10 wt %.

10. The method of claim 8, wherein the concentration of magnetic sensitive Hematite (α-Fe₂O3) is 15 wt %.

11. The method of claim 8, wherein the concentration of magnetic sensitive Hematite (α-Fe₂O₃) is 20 wt %.

12. The method of claim 8, further comprising:
configuring the stent to have a desired magnetically induced shape memory position.

13. A method of using a patient-specific stimuli-responsive cardiovascular stent for coronary artery spasm treatment to eliminate post-surgical interventions comprising:
determining a desired configuration of the stimuli-responsive cardiovascular stent based on a physiology of a patient; and
producing a stimuli-responsive cardiovascular stent based on the desired configuration.

14. The method of claim 13, wherein the cardiovascular stent is produced by a process of additive manufacturing.

15. The method of claim 14, wherein the process of additive manufacturing occurs within the vicinity of the patient.

16. The method of claim 13, wherein the cardiovascular stent comprises a PLA/α-Fe₂O₃ nanocomposite.

17. The method of claim 16, wherein the concentration of α-Fe₂O₃ in the PLA/α-Fe₂O₃ nanocomposite is at least 10 wt %.

18. The method of claim 16, wherein the concentration of α-Fe₂O₃ in the PLA/α-Fe₂O₃ nanocomposite is at least 15 wt %.

19. The method of claim 16, wherein the concentration of α-Fe₂O₃ in the PLA/α-Fe₂O₃ nanocomposite is at least 20 wt %.

20. The method of claim 13, further comprising:
providing the cardiovascular stent to the patient; and
actuating the cardiovascular stent by applying a magnetic field near the cardiovascular stent.
